(19) United States
(12) Patent Application Publication
(10) Pub. No.: US 2016/0208240 A1
(43) Pub. Date: Jul. 21, 2016

(54) NGS WORKFLOW

(71) Applicant: VELA OPERATIONS PTE.LTD., Singapore (SG)

(72) Inventors: Rui ZHANG, Singapore (SG); See Ting LEONG, Singapore (SG); Rouh San SIOW, Singapore (SG); Arseny SMIRNOV, Singapore (SG); Chao Ping LOU, Singapore (SG); Yong Qiang YEO, Singapore (SG)

(21) Appl. No.: 14/897,975

(22) PCT Filed: Jun. 12, 2014
(86) PCT No.: PCT/IB2014/062174

§ 371 Date: Dec. 11, 2015

(30) Foreign Application Priority Data
Jun. 13, 2013 (EP) 13171793.6

(51) Int. Cl. C12N 15/10; C12Q 1/68

(52) U.S. Cl. CPC C12N 15/1093; C12Q 1/6874; C12N 15/1096; C12Y 302/02027; C12Y 207/07007; C12Y 207/07049

(57) ABSTRACT

The present invention relates to improved semi-automated methods that permit the extraction of nucleic acids from samples, preparation of PCR and post-PCR preparation steps of DNA-libraries for next-generation sequencings methods that can be conducted. The methods and additional aspects relating to such methods are less laborious, safe costs, reagents and are less prone to contamination than comparable methods that are not automated.

### BACKGROUND INFORMATION

The present invention relates to the field of nucleic acid sequence analysis. In particular, the present invention relates to methods and tools relating to Next-Generation Sequencing (NGS). DNA sequencing is a powerful approach for decoding a number of human diseases, including different types of genes involved in the development of cancers.

The advent of next-generation sequencing (NGS) technologies has reduced sequencing cost by orders of magnitude and significantly increased the throughput, making whole-genome sequencing a possible way for obtaining global genomic information about patients on whom clinical actions may be taken. DNA sequencing may be used to determine the sequence of individual genes, larger genetic regions (i.e. clusters of genes or operons), full chromosomes or entire genomes. Depending on the methods used, sequencing may provide the order of nucleotides in DNA or isolated from cells of animals, plants, bacteria, etc., or virtually any other source of genetic information. The resulting sequences may be used by researchers in molecular biology or genetics and to further scientific progress or may be used by medical personnel to make treatment decisions or aid in genetic counseling. The latter two uses are often cited in the context with personalized medicine or companion diagnostic applications.

Irrespective of benefits offered by NGS technologies a number of challenges that must be adequately addressed before they can be transformed from research tools to routine clinical practices. NGS technologies for diagnostic purposes should require as little manual steps, include adequate mechanisms for preventing contamination by nucleic acid material originating from other sources than the clinical sample that is subject to analysis at a given time point, and the methods should be fast and should be easily performed by staff working in a clinical laboratory.

Different NGS techniques have been developed, which involve physico-chemical mechanisms resulting in distinct methods used in the analysis of respective nucleic acid sequences. These techniques are generally known in the technical field. The most widely applied techniques are Ion semiconductor (Ion Torrent) sequencing, pyrosequencing and sequencing by synthesis (Illumina).

### DEFINITIONS

Having described the method of the invention generally, each aspect of this method will be described in greater detail.

As used herein, the nucleic acid being sequenced is referred to as the target nucleic acid (or the target). Target nucleic acids include but are not limited to DNA such as but not limited to genomic DNA, mitochondrial DNA, cDNA and the like, and RNA such as but not limited to mRNA, miRNA, and the like. The target nucleic acid may derive from any source including naturally occurring sources or synthetic sources. The nucleic acids may be PCR products, cosmids, plasmids, naturally occurring or synthetic library members or species, and the like. The invention is not intended to be limited in this regard. The nucleic acid may be from animal or pathogen sources including without limitation mammals such as humans, and microbes such as bacteria, viruses, fungi, parasites, and mycobacteria. In some embodiments, the nucleic acid is not a viral nucleic acid. The target nucleic acid can be obtained from any bodily fluid or tissue including but not limited to blood, saliva, cerebrospinal fluid (“CSF”), skin, hair, urine, stool, and mucus. The target nucleic acid may also be derived from without limitation an environmental sample (such as a water sample), a food sample, or a forensic sample, the sample may be a fresh sample (e.g. biopsy material directly subjected to nucleic acid extraction), or a sample that has been treated to allow storage, e.g. a sample that was formalin-fixed and/or paraffin-embedded (FFPE samples).

Target nucleic acids are prepared using any manner known in the art. As an example, genomic DNA may be harvested from a sample according to techniques known in the art (see for example Sambrook et al. “Maniatis”). Following harvest, the DNA may be fragmented to yield nucleic acids of smaller length. The resulting fragments may be on the order of hundreds, thousands, or tens of thousands of nucleotides in length. In some embodiments, the fragments are 50-1000 nucleotides in length, 100-1000 nucleotides in length, 200-1000 base pairs in length, or 300-800 base pairs in length, although they are not so limited. Nucleic acids may be fragmented by any means including but not limited to mechanical, enzymatic or chemical means. Examples include shearing, sonication, nebulization and endonuclease (e.g., DNase I) digestion, or any other technique known in the art to produce nucleic acid fragments, preferably of a desired length. Fragmentation can be followed by size selection techniques used to enrich or isolate fragments of a particular length. Such techniques are also known in the art and include but are not limited to gel electrophoresis or SPRI.

Alternatively, target nucleic acids that are already of a desired length may be used. Such target nucleic acids include those derived from an exon enrichment process. See Albert et al. Nat Meth 4(11):903-905 (2007), Porreca et al. Nat Meth 4(11):931-936 (2007), Okou et al. Nat Meth 4(11):907-909 (2007) for methods of isolating and/or enriching sequences such as exons prior to sequencing. Thus, rather than fragmenting (randomly or non-randomly) longer target nucleic acids, the targets may be nucleic acids that naturally exist or can be isolated in shorter, useable lengths such as mRNAs, cDNAs, exons, PCR products (as described above), and the like.

Generally, the target nucleic acids are ligated to sequences on one or both the 5′ and 3′ ends. These adaptor sequences comprise sequencing primer sites (i.e., sites to which a sequencing primer will hybridize) to be used in the sequencing methods of the invention.

In some embodiments, the targets subjected to amplification, as discussed below, are of the same or similar length (e.g., a 5-10% variation between targets). In some embodiments, such variation may be kept as small as possible in order to ensure that all templates are uniformly applied.

The amplified products can be immobilized to the support surface (e.g., a glass surface) in a variety of ways. For example, the amplification process may be carried out in solution and the final product is then attached to the support surface. The amplification product may be attached to the solid support at its 5′ end or its 3′ end. Attachment may be through hybridization to a nucleic acid that is immobilized to the support surface or it may be through interaction of moieties on the end of the amplification product with moieties on the support surface. Examples include the use of biotin or dual biotin labelled DNA (Margulies et al. Nature 437:376 (2005)) with streptavidin/avidin/neutravidin coated support surfaces, DIG (digoxigenin) and anti-DIG antibodies or antibody fragments, fluorescein and anti-fluorescein antibodies or antibody fragments (Gore et al. Nature 442, 836-9 (2006)), or through the use of heterofunctional cross-linkers such as biotinylated succinimidyl propionate-PEG which can be coupled for example to amine-functionalized glass and used to immobilize biotin-labelled DNA through a streptavidin sandwich (i.e., a nucleic acid biotin streptavidin/avidin/neutravidin-biotin solid support interaction).

The templates may be referred to as being randomly immobilized onto the surface. This means that the templates are not placed on the solid support surface based on sequence. They are however placed on the solid support in a manner that ensures that each template is surrounded by an area (and thus volume) that will not be occupied by another template during the polymerase-mediated incorporation reactions and/or during extension of the template. That is, in some instances, the templates are positioned on the surface at a sufficient distance from each other to prevent any interaction between the templates.

The solid support refers to the element to which the template is bound or immobilized can be comprised of any material, including but not limited to glass or other silica based material, plastic or other polymer based material, provided however that the material is relatively inert to template, primer, polymerase, dNTPs, and other components used in the sequencing reaction and wash. The solid support may or may not be rigid. It may be porous. It may or may not be continuous. In some embodiments, the solid support is a glass slide. In some embodiments, the support is a plurality of beads or particles (such as microparticles) that are themselves immobilized onto a solid support. Such beads may be porous. The support may be a mesh. In some embodiments, the solid support is itself a detector or a sensor such as but not limited to a contact imager.

It is to be understood that a plurality of templates whether identical or different may be tethered to the solid support, provided that each member of the plurality is sufficiently spaced apart from other members so that no overlap occurs between templates.

Typically, the template must be attached to an observable (or detectable) moiety on its free end. This moiety is intended to represent the free end of the template and thus its position and movement in the direction of the force indicates the length of the template. The observable moiety can be any number of moieties and the invention is not limited by its nature. The nature of the observable moiety will dictate the type of sensor or detector suitable to observe (or detect or monitor) changes in the length of the template. In some important embodiments, the observable moiety is a bead such as a microbead, and even more particularly such as a magnetic bead.

The moieties can be attached to the template through a variety of methods and employing a variety of interactions, including but not limited to non-covalent interactions such as biotin/streptavidin, DIG/anti-DIG, and fluoroscein/anti-fluoroscein binding pairs, as well as covalent interactions, such as those discussed herein in relation to covalent immobilization of templates (or primers) to support surfaces.

The solid support is part of or adjacent to a flow cell. As used herein, a flow cell is a chamber having at least an inlet and an outlet port through which a fluid travels. The solid support to which the template is tethered may be below, above or beside the flow cell, depending on the position of the detection system used to observe the template. The solid support may be a wall of the flow cell including a bottom wall, a side wall, or a top wall.

As will be appreciated, accurate and rapid sequencing of the template is dependent on the extent to which and the rate at which unincorporated nucleotides are removed from the system. Thus, rapid and complete (or near complete) removal of unincorporated nucleotides is important. The microfluidic system must also be designed to maximize washing potentially resulting in smaller wash volumes and wash duration.

Clearance of unincorporated nucleotides can also be facilitated in part or in whole through the use of apyrase which degrades unincorporated dNTPs and renders them unsuitable for further incorporation. The apyrase may be free flowing, added to the wash buffer, and introduced into the flow cell once incorporation of any given nucleotide triphosphate type has ceased (as indicated by the cessation of any above-background movement by the detectable moiety at the end of the template). Alternatively or additionally, apyrase may be fixed or immobilized within the flow cell such as for example to the solid support surface (to which the template is also fixed or immobilized). This may occur through the use of a linker in order to make the enzyme more accessible and to remove any steric hindrance relating to close proximity to the surface. Apyrase may be attached to a variety of linkers that differ in length. In this way, apyrase may be present in a variety of flow streams within the flow cell, including those closer to the walls and those that are closer to or at the center flow streams. As discussed above, it is the flow streams near the walls which travel with low velocity and unincorporated dNTPs present in these flow streams are less likely to be cleared away. Having apyrase in these flow streams should improve removal of these dNTPs. This will increase the likelihood that changes in template length are a result of incorporation of a dNTP newly introduced into the flow cell rather than a residual and unincorporated dNTP that remains in the flow cell after washing.

In some aspects of the invention, the sequencing methods are referred to as sequencing-by-synthesis reactions. This means that determining the sequence of a first nucleic acid requires the synthesis of a second nucleic acid using the first as a template. In this way, the sequence of the second nucleic acid is determined from the order and number of incorporated dNTPs, and the sequence of the first nucleic acid is determined as the complement of the first nucleic acid sequence. The methods of the invention detect dNTP incorporation by a change in length of the template and not by directly observing the addition of the dNTP to nucleic acid being synthesized. As a result, the dNTP can be natural dNTP (i.e., dNTP that lack any modification including any exogenous detectable label such as a fluorophore). As should be clear from this disclosure, the sequencing methods of the invention also require that the template remains intact. Some aspects of the invention involve sequencing methods that are described as occurring in the absence of fluorescence or in a non-fluorescent manner. These characterizations mean that the methods can be carried out without detection of fluorescence, particularly without detection of fluorescence from each incorporated dNTP. Embodiments of these methods therefore may employ natural dNTPs that have not been modified by addition of an exogenous fluorophore. These characterizations do not exclude however the possibility that the observable moiety conjugated to the free end of the template is itself fluorescent. In this latter instance, changes in the length of the template may be visualized via the fluorescence of the observable moiety rather than any fluorescence from individually incorporated dNTP.

Similarly, it will also be understood that the sequencing methods provided herein are able to detect nucleotide incorporation by detecting the observable moiety itself (e.g., as is possible with a CMOS contact imager). Thus, in some embodiments, the observable moieties are detected directly and without the need for an enzyme-mediated event. An example of enzymatically detected nucleotide incorporation is pyrosequencing coupled with sulfurylase and luciferase mediated detection of released inorganic pyrophosphate. (See Leamon and Rothberg, Chemical Reviews, “Cramming More Sequencing Reactions onto Microreactor Chips”, 2006.) Thus, aspects of the invention are referred to as non-enzymatic methods (or as detecting nucleotide incorporation non-enzymatically) since nucleotide incorporation can be detected in the absence of enzyme-generated signals.

In various embodiments, an analyte of particular interest is hydrogen ions, and large scale ISFET arrays according to the present disclosure are specifically configured to measure pH. In other embodiments, the chemical reactions being monitored may relate to DNA synthesis processes, or other chemical and/or biological processes, and chemFET arrays may be specifically configured to measure pH or one or more other analytes that provide relevant information relating to a particular chemical process of interest. In various aspects, the chemFET arrays are fabricated using conventional CMOS processing technologies, and are particularly configured to facilitate the rapid acquisition of data from the entire array (scanning all of the pixels to obtain corresponding pixel output signals). A preferred sequencing system is the Ion PGM System, however, other sequencing system based on proton detection are also contemplated. For example, pyrosequencing systems and Illumina sequencing-by-synthesis are options. With respect to analyte detection and measurement, it should be appreciated that in various embodiments discussed in greater detail below, one or more analytes measured by a chemFET array according to the present disclosure may include any of a variety of chemical substances that provide relevant information regarding a chemical process or chemical processes of interest (e.g., binding of multiple nucleic acid strands, binding of an antibody to an antigen, etc.). In some aspects, the ability to measure levels or concentrations of one or more analytes, in addition to merely detecting the presence of an analyte, provides valuable information in connection with the chemical process or processes. In other aspects, mere detection of the presence of an analyte or analytes of interest may provide valuable information. The most preferred sequencing method of the present invention involves the use of Ion Torrent's PGM System.

In another aspect, the invention provides a method for sequencing nucleic acids comprising fragmenting a template nucleic acid to generate a plurality of fragmented nucleic acids, attaching one strand from each of the plurality of fragmented nucleic acids individually to beads to generate a plurality of beads each having a single stranded fragmented nucleic acid attached thereto, delivering the plurality of beads having a single stranded fragmented nucleic acid attached thereto to a chemFET array having a separate reaction chamber for each sensor in the area, and wherein only one bead is situated in each reaction chamber, and performing a sequencing reaction simultaneously in the plurality of chambers.

The invention contemplates performing a plurality of different sequencing reactions simultaneously within the same flow cell or on the same solid support. Each sequencing reaction yields information about one template immobilized on the solid support. The number of templates that can be sequenced in a single run will depend on the expected length of the template and the area of the solid support. Therefore depending on the embodiment, at least 100, at least 200, at least 300, at least 400, at least 500, at least 600, at least 700, at least 800, at least 900, or at least 1000 templates may be immobilized on a solid support and thus sequenced simultaneously. In still other embodiments, 100-500, 100-750, 100-1000, 500-1000, 600-1000, 700-1000, 800-1000, 900-1000, 1000-2000, 2000-3000, 3000-4000, 4000-5000, 5000-10000, or more templates may be sequenced simultaneously. Table 1 shows that the solid support can be configured to have 1.6 pixels per 2.8 μm bead.

The sequencing reaction is carried out by incorporating dNTPs into a newly synthesized nucleic acid strand that is hybridized to the template. The newly synthesized strand may derive from a primer that is bound to the template or from other molecule from which polymerase-mediated extension can proceed.

In one non-limiting example, the sequencing reaction may be commenced by contacting templates with primers under conditions that permit their hybridization, and contacting template/primer hybrids with polymerases. Such contacting may occur before, during and/or after immobilization to the solid support. In an important embodiment, it occurs following immobilization to the solid support.

Once the primers and polymerases are bound to the template, repeated cycles of reagents are flowed into and through the flow cell. When the reagent flow contains a nucleotide that is complementary to the nucleotide on the template that is directly downstream of the 3′ end of the primer, the polymerase will incorporate the dNTP. If contiguous downstream positions on the template are occupied by identical nucleotides (referred to herein as a homopolymer), the polymerase will incorporate an identical number of complementary dNTPs. Such incorporation will cease when the dNTP in flow is not complementary to the next available nucleotide on the template. The amount of flowed dNTP and the time of such flow will respectively exceed the number of complementary bases on the template and the time needed to incorporate all possible dNTPs.

Importantly, incorporation of the complementary dNTPs occurs at more than one of the bound primers. More preferably, incorporation occurs at least 10%, at least 25%, at least 50%, at least 60%, at least 70%, at least 80%, at least 90%, or at all of the bound primers. The percentage of primers may depend upon the number of target copies in the template. For some embodiments, incorporation occurs at least 30, at least 35, at least 40, at least 45, at least 50, at least 60, at least 70, at least 80, at least 90, at least 100 or more primers per individual template. It will be understood that the invention contemplates incorporating dNTPs at as many of the hybridized primers on a given template in order to increase signal to noise ratio by increasing the magnitude of the length change that occurs (whether it is an increase or decrease in length).

As part of the sequencing reaction, a dNTP will be ligated to (or “incorporated into” as used herein) the 3′ of the newly synthesized strand (or the 3′ end of the sequencing primer in the case of the first incorporated dNTP) if its complementary nucleotide is present at that same location on the template nucleic acid. Incorporation of the introduced dNTP converts a single stranded region of the template into a double stranded region, and this conversion is then reflected in a change in length of the template under tension. The change in length is detected by determining and monitoring the position of the observable moiety (e.g., a bead) located at the free end of the template. Therefore, if the bead position is unchanged after any given flow through, then no dNTPs have been incorporated and one can conclude that the flow through dNTP was not complementary to the next available nucleotide in the template. If a change in position of the moiety is detected, then the flow through dNTP was complementary and was incorporated into the newly synthesized strand. dNTPs may be flowed in any order provided the order is known and is preferably kept constant throughout the sequencing run.

A typical sequencing cycle for some aspects of the invention may include washing of the flow chamber (and wells) with wash buffer, measurement of the position of the observable moiety tethered to the end of the template nucleic acid, introduction of a first dNTP species (e.g., dATP) into the flow chamber in the presence of polymerase, measurement of the position of the observable moiety, flow through of apyrase optionally in wash buffer, flow through of wash buffer, introduction of a second dNTP species in the presence of polymerase, and so on. This process is continued until all 4 dNTP (i.e., dATP, dCTP, dGTP and dTTP) have been flowed through the chamber and allowed to incorporate into the newly synthesized strands. This 4-nucleotide cycle may be repeated any number of times including but not limited to 10, 25, 50, 100, 200, 300, 400, 500, 600, 700, 800, 900, 1000 or more times. The number of cycles will be governed by the length of the target being sequenced and the need to replenish reaction reagents, in particular the dNTP stocks and wash buffers. Thus, the length of sequence that may be determined using the methods of the invention may be at least 50 nucleotides, at least 100 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, up to and including 1000 nucleotides, 1500 nucleotides, 2000 nucleotides or more nucleotides

Suitable polymerases can be DNA polymerases, RNA polymerases, or subunits thereof, provided such subunits are capable of synthesizing a new nucleic acid strand based on the template and starting from the hybridized primer. An example of a suitable polymerase subunit is the exo-version of the Klenow fragment of E. coli DNA polymerase I which lacks 3′ to 5′ exonuclease activity. Other suitable polymerases include T4 exo-, Therminator, and Bst polymerases. The polymerase may be free in solution (and may be present in wash and/or dNTP solutions) or it may be fixed to the solid support, one or more walls of the flow cell, the template, or the primers.

It will be understood that the sequencing methods provided herein have a number of applications including without limitation determining partial or complete nucleotide sequence of a nucleic acid (or a collection of nucleic acids such as exist in a genome, including mammalian genomes and more particularly human genomes), determining the presence or absence of a nucleic acid in a sample (as can be useful in for example diagnostic and forensic methods), determining whether the nucleic acid comprises a mutation or variation in sequence (such as for example an allelic variation including a single nucleotide polymorphism), determining whether a known nucleic acid has undergone mutation resulting in the generation of a new species (such as may be the underlying cause of antibiotic resistant microorganisms), determining the presence of a genetically modified organism or genetically engineered nucleic acids, determining whether and what genetic differences exist between two samples (such as for example normal tissue and diseased tissue), determining what therapeutic regimen will be most effective to treat a subject having a particular condition as can be determined by the subject's genetic make-up, and genotyping (e.g., analyzing one or more genetic loci to determine for example carrier status). In some of these embodiments, the nucleotide sequence determined using the methods of the invention may be compared to a known or reference sequence in order to orient the obtained sequence and/or to identify differences between the two. This may help to identify genetic variation and mutation. The known or reference sequence may be a previously determined sequence (for example, resulting from the complete genomic sequencing of a species).

The methods described herein can also be used to aid in the identification and treatment of condition. For example, the methods can be used for identifying a sequence associated with a particular condition or for identifying a sequence that is used to diagnose the absence of a particular condition. The samples being analyzed may be from any subject including humans. The condition may be cancer or an infection.

The methods can also be used to identify a sequence associated with a positive response to an agent. The method may comprise sequencing DNA from a plurality of subjects that exhibited a positive response and from a plurality of subjects that exhibited a negative response to an agent using one or more sequencing methods provided herein, and identifying a common sequence in the plurality of subjects that exhibited a positive response or from the subjects that exhibited a negative response that this sequence is not present in the other plurality of subjects. Preferably, the subject is a mammal, and more preferably a human.

The methods described herein may be automated such that the sequencing reactions are performed via robotics. In addition, the sequencing data obtained from a detector or a sensor may be input to a personal computer, a personal digital assistant, a cellular phone, a video game system, or a television, so that a user can monitor the progress of the sequencing reactions remotely.

The invention further contemplates kits comprising the various reagents necessary to perform the amplification and/or sequencing reactions and instructions of use according to the methods set forth herein.

The methods provided herein are dependent upon detecting single nucleotides at each copy of a target in the template. The limit of resolution is dependent upon the resolution of the detection system used.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

As used in this specification and the appended claims, the singular forms “a,” “an,” and “the” include plural referents unless the context clearly dictates otherwise.

### EMBODIMENTS OF THE PRESENT INVENTION

The present invention relates, amongst others, to unique semi-automated methods for the isolation of nucleic acids from samples, set-up of (RT-)PCR reaction, (RT-)PCR-based nucleic acid amplification, post-PCR normalization and clean up of amplification products, fragmentation of PCR amplification products, ligation with adaptors characterized by the following steps set out in (A) and (B):

### Method (A)

(a) Extraction of nucleic acids from a sample;
(b) Optionally addition of Uracil-DNA-glycosylase (UDG) to the (RT-) PCR mixture before conducting (RT-) PCR reaction to digest cross and carryover contamination from prior amplification reactions;
(c) (RT-)PCR, depending on type of isolated nucleic acids, i.e. RNA or DNA, using nucleotide triphosphate building blocks (i.e. individual nucleotides) comprising A, T, C, G, optionally also comprising Uracil;
(d) Normalization of nucleic acids obtained in RT-PCR (using carrier structures, e.g. paramagnetic microbeads (e.g. AxyPrep Mag PCR Normalizer, Axygen) for normalization, wherein said beads bind nucleotide sequences of a desired sequence length) comprising binding RT-PCR mixture subsequent to PCR to said beads, thoroughly washing the microbeads subsequent to binding of PCR-product, elution of PCR amplification products from microbeads;
(e) Fragmentation (Shearing) eluted PCR amplification products obtained in step (d);
(f) Binding the product of step (e) to carrier structures, e.g. microbeads, followed by washing and elution of the bound nucleic acids;
(g) Ligation of adaptor sequences (comprising barcode sequences allowing attribution of nucleic acids to specific sample (e.g. clinical sample and patient) to the product obtained in step (f);
(h) Cleaning up the product obtained in step (g) using carrier structures, e.g. microbeads used in previous steps (d) and/or (f);
(i) Subjecting the product obtained in step (h) to sequencing reaction (e.g. using Ion PGM System), and
(j) Analysis of the results of the sequencing reaction obtained in step (i).

### Method (B)

(a) Extraction of nucleic acids from a sample;
(b) Optionally addition of Uracil-DNA-glycosylase (UDG) to the (RT-) PCR mixture before conducting (RT-) PCR reaction to digest cross and carryover contamination from prior amplification reactions;
(c) RT-PCR, depending on type of isolated nucleic acids, i.e. RNA or DNA, using nucleotide triphosphate building blocks (i.e. individual nucleotides) comprising A, T, C, G, optionally also comprising Uracil;
(d) Partial digestion of primers (e.g. using FuPa reagent of Life Technologies);
(e) Ligation of adaptor sequences (comprising barcode) to the product obtained in step (d);
(f) Normalization of nucleic acids obtained in RT-PCR (using carrier structures, e.g. paramagnetic microbeads (e.g. AxyPrep Mag PCR Normalizer, Axygen) for normalization, wherein said beads bind nucleotide sequences of a desired sequence length) comprising binding RT-PCR mixture subsequent to PCR to said beads, thoroughly washing the microbeads subsequent to binding of PCR-product, elution of PCR amplification products from microbeads;
(g) Clean up product obtained in step (g) using carrier structures, e.g. microbeads used in previous steps (d) and/or (f);
(i) Subject product obtained in step (h) to sequencing reaction (e.g. using Ion PGM System; Ion Torrent), and
(j) Analysis of the results of the sequencing reaction obtained in step (i).

The uniqueness of the above workflow methods allows reducing the amount of time required in the process from the extraction of the nucleic acids for analysis and the final NGS reaction, which is followed by analysis of the results. The use of UDG largely reduces the risk of contamination in automated systems for nucleic acid extraction, PCR set-up, post-PCR purification steps, library preparation and NGS. Automation of these steps using the above methods reduces the time and costs required in particular for diagnostic applications.

Surprisingly, it was noticed that an innovative alternative method described herein can be used in preparation of next-generation sequencing libraries. The inventive methods can be used in the preparation of different types of NGS-libraries, e.g. for Illumina sequencing or for Ion Torrent sequencing. This method can be incorporated into the NGS workflow set out above.

Usually, NGS libraries are prepared using commercially available kits including buffers that are suitable for such purpose. These buffers are specifically optimized for the robust, high-fidelity amplification of NGS-libraries, regardless of the GC-content. As automated open systems for the preparation of NGS libraries can be susceptible to the high risk for the carry-over of contamination of clinical samples by PCR amplicons from previous runs, it is an objective to reduce said danger. To prevent carry-over contamination, dUTP is added to (RT-) PCR master mixes. Uracil dehydrogenase (UDG)-treatment of PCR master mixes removes contaminant amplicons from previous runs and that may accidentally have been carried over into subsequent reaction mixtures. Uracil dehydrogenase (UDG) is an enzyme that removes uracil from DNA by hydrolysis of the N-glycosylic bond between the deoxyribose and the base leaving an apurinic or apyrimidinic site (AP site).

However, buffers for NGS-library preparation (e.g. SuperScript™ III One-Step RT-PCR System with Platinum® Taq High Fidelity) generally are not suitable for the incorporation of dUTP during amplification reactions. It was surprisingly noticed that specific high fidelity enzymes specifically developed for NGS-library preparation can be replaced by conventional Taq Polymerase, which are non-high fidelity enzymes. Conventional (RT-)PCR buffer (e.g. buffers containing 100 mM Tris-HCl, pH 8.3, 500 mM KCl, 15 mM MgCl₂) can be used. This modification in the protocol for preparation of NGS-libraries allows incorporation of dUTP during amplification.

Accordingly, in one aspect the present invention relates to methods of preparing NGS libraries comprising incorporation of dUTP during amplification without using specialized high fidelity PCR buffers, but wherein essentially any DNA polymerase (e.g. Taq Polymerase) that is suitable for PCR is used. This rather simple exchange of buffers and enzymes allow the introduction of dUTP and subsequent treatment with UDG to prevent carry-over of contaminants.

Further, the invention relates to a method for elimination of carry-over contamination, i.e. for the decontamination of reagent mixtures comprising extracted nucleic acids that should be analysed and potentially contaminating DNA derived from previous (RT-)PCR reactions, in nucleic acid amplification reactions for the preparation of a next generation sequencing library using wild-type (recombinant or native) Taq polymerase for the incorporation of dUTP, comprising the steps of:
a) fragmenting nucleic acids obtained from a sample,
b) adding a degrading enzyme suitable to degrade any contaminating nucleic acid amplificates present in the amplification reaction mixture;
c) amplifying a nucleic acid template in order to provide a first nucleic acid amplificate in a first nucleic acid amplification reaction in the presence of dUTP; and
d) inactivating said degrading enzyme.

In a preferred embodiment of the above method for elimination of carry-over contamination in nucleic acid amplification reactions for the preparation of a next generation sequencing library using wild-type (recombinant or native) Taq polymerase or a derivative thereof for the incorporation of dUTP, the degrading enzyme is UDG. The UDG treatment usually takes several minutes, e.g. up to 10 minutes, preferably up to 5 minutes. Subsequently, the enzyme is deactivated, e.g. by exposure to temperatures of about 50° C. for about 5 minutes.

In another preferred embodiment of the above method for elimination of carry-over contamination in nucleic acid amplification reactions for the preparation of a next generation sequencing library using wild-type Taq polymerase for the incorporation of dUTP, the degrading enzyme is UDG the Taq Polymerase is recombinant or native polymerase.

In preferred embodiment of the above method for elimination of carry-over contamination in nucleic acid amplification reactions for the preparation of a next generation sequencing library using wild-type (recombinant or native) Taq polymerase for the incorporation of dUTP, the degrading enzyme is UDG, and the UDG-treated library is subjected to further steps in the next generation sequencing method, comprising:
a) fragmenting nucleic acids obtained from a sample,
b) adding a degrading enzyme suitable to degrade any contaminating nucleic acid amplificates present in the amplification reaction mixture;
c) amplifying a nucleic acid template in order to provide a first nucleic acid amplificate in a first nucleic acid amplification reaction in the presence of dUTP; and
d) inactivating said degrading enzyme.

Preferred embodiments of the present methods for the generation of DNA libraries, or the decontamination of reaction mixtures in the process of the above DNA library preparation are also depicted in the claims.

Preferred embodiments of methods (A) and (B) above relate to in vitro diagnostic applications, e.g. in companion diagnostics where knowledge about the sequence of a target nucleic acid (for example, an oncogene or a nucleic acid derived from a pathogen like HCV, HIV, or the like) present in a clinical sample helps the physician to select the most promising treatment for a patient, because modifications in some oncogenes confer resistance to certain drugs.

In a preferred embodiment of method (A), the sample is a fresh sample obtained, e.g. from a patient, preferably a human patient. The sample material may be, for example, blood, plasma, a subpopulation of blood cells, e.g. T-cells, cerebrospinal fluid, sputum, stool, and the like.

In a preferred embodiment of method (A), the sample is plasma in order to isolate nucleic acid material found therein, e.g. viral, bacterial, fungal, or parasite-derived nucleic acids or material containing such nucleic acids, e.g. virions, bacteria, and the like.

In a preferred embodiment of method (A) the sample material is plasma and the nucleic acid material is derived from a virus, e.g. HCV, HIV, etc. When HCV is targeted, the region of interest is preferably the NS5B gene region, which is well-suited to identify 6 major HCV genotypes and a large number of subtypes. The target region in of the HCV genome is preferably extending from nucleotide 8616 to nucleotide 9298, but the region may be slightly longer or shorter as long as the identification of 6 HCV genotypes is possible. Preferred primers bind to nucleotides 8616-8638, 8614-8635, 9276-9298 and 9171-9191 of the HCV genome. The primers may comprise natural or modified nucleotide building blocks as known in the art.

In a preferred embodiment of method (B), the sample is a fresh sample obtained, e.g. from a patient, preferably a human patient. The sample material may, for example, be blood, plasma, a subpopulation of blood cells, e.g. T-cells, cerebrospinal fluid, sputum, stool, and so forth. In another preferred embodiment of method (B), the sample is not a fresh sample, but a sample that has been treated after obtaining the same, e.g. using formalin-fixation and/or paraffin-embedding (FFPE samples are preferred samples for analysis of various oncogenes).

In a preferred embodiment of method (B), the sample material is an FFPE-sample derived from a human patient, e.g. a sample from any tissue that may be formalin-fixed and/or paraffin-embedded, e.g. a sample derived from skin, breast tissue, colon, lung, liver, muscle, etc. In a very preferred embodiment, the sample is skin sample for analysis of genes involved in melanoma formation. Preferred genes targeted in this context comprise at least one or more of the following group of genes: NRAS, AKT3, MAP2K1, GNA11, ERBB4, PIK3CA, FGFR3, KIT, BRAF, CDKN2A, and GNAQ. These genes are known to be involved in the development of melanoma and may contain different point mutations at different sites of the respective genes. The analysis of specific mutations allows the treating physician to choose a suitable therapy as some mutations are known to confer drug resistance, whereas others are drugable (sensitive to drugs).

Another aspect of the present invention is the provision of new FFPE cell lines that may serve as control material for nucleic acid extraction from FFPE tissue. These cell lines may carry genetic information that corresponds to the targeted sequence, e.g. genetic material that was previously introduced via transformation or using other methods. Alternatively, these genes may not have been genetically modified, e.g. when the cells already carry target genes of interest (for example oncogenes) or when the target gene should be different from the gene targeted in the actual assay. For example, when the assay targets mutations of one or more oncogenes in clinical sample, the gene targeted in the FFPE cell lines may be a house-keeping gene, or a non-mutated wildtype gene. The cell lines provide a source of quantifiable amounts of target nucleic acid, since the amount of FFPE cell line material may be selected to match the requirements of individual assays. The inventive cell lines may be provided as a part of a kit for any given assay. Said kit may further comprise additional chemical reagents suitable for the extraction, purification, amplification or other manipulation of nucleic acids, e.g. primers, buffers, enzymes, and the like.

Another aspect provided herein is a method for the normalization of DNA libraries. In further embodiments, these DNA libraries are used for subsequent NGS involving the use of carrier particles such as magnetic microbeads.

In prior methods, the normalization of DNA libraries required the quantification and/or size selection of fragmented DNA amplification products obtained in (RT-) PCR reactions before ligation of adapters. It was surprisingly found out that the library preparation involving the use of microbeads does not require size selection and/or prior quantification, preferred microbeads are those provided by Axygen (AxyPrep MAG-PCR-CL-5Kit) or similar products. The use of these microbeads eliminates also shorter fragments still present after nucleic acid amplification and/or ligation of adapters to the amplification products.

Furthermore it was surprisingly found out that the PCR amplification of thus generated DNA libraries is not necessary, unlike in prior art methods where the library comprising adapters subsequent to ligation was amplified again.

Depending on the quantity of beads and the incubation time of said beads with the DNA library, the quantity of bound DNA can be defined, since the beads are saturated with nucleic acids over time.

The inventive automated nucleic acid extraction, amplification and library preparation method (e.g. using the Sentosa SX101 platform of Vela Diagnostic) allows reducing time, amount of reagents and costs in general and avoids the risk associated with manual preparation of DNA libraries for NGS.

The present invention also contemplates a kit for the preparation of generic libraries.

Still further, the present invention provides a simplified and improved library preparation protocol. As mentioned above, normalizing magnetic beads for the preparation of DNA libraries that are used the subsequent NGS protocol are very important in order to obtain correct amounts of nucleic acids for further analysis. To this end, DNA binding beads with limited binding surface can be used after (RT-)PCR can be used for normalization of the amplified nucleic acids.

Further, to obtain a pre-defined amount of DNA for the following next generation sequencing steps, prior art methods essentially required the following steps:
1) (RT-)PCR
2) Clean-up of PCR products using magnetic beads and clean-up buffer
3) Washing the beads (e.g. with ethanol)
4) Elution of PCR product bound to magnetic beads
5) Normalization of PCR products using normalization magnetic beads and normalization buffer
6) Washing the beads (e.g. with ethanol)
7) Elution of normalized PCR product.

Normalization magnetic beads (Definition) are very sensitive to RT-PCR buffer, presumably because dTT in one-step RT-PCR buffers inhibit the binding of amplified DNA products to normalization beads. It was previously necessary in prior art methods to perform the above steps 2) to 4), which remove reagents present in RT-PCR mixture after amplification was carried out.

Surprisingly, the present inventors found out that tedious, time-consuming and costly steps 2) to 4) can be omitted when the (RT-)PCR products are exposed to a new inventive composition comprising for normalization beads for NGS library preparation comprising a solvent, e.g. polyethylene glycol and an alkali metal salt, e.g. NaCl, MgCl, or the like. In some embodiments, the composition comprises, e.g. about 2.0 to about 5.0 M NaCl, e.g. 2.0 M to about 4.0 M NaCl, preferably 2.5 M to about 3.5 M NaCl, very preferably about 2.5 to about 3.0 M NaCl, and most preferably the concentration of the alkali metal in the inventive buffer is 2.5 M NaCl. The inventive buffers for normalization beads for NGS library preparation further comprises about 10% to about 30% of a solvent, e.g. about 12.5% to about 25%, or 15.0% to about 25%, or 17.5% to about 22.5%, preferably about 20% of a solvent. The solvent is preferably a polyethylenglycol, e.g. high molecular weight PEG such as Polyethylenglycol (PEG) 8000. It is possible also to replace NaCl by other alkali metal salts such as Mg, K, etc. In a very preferred embodiment the inventive buffers for normalization beads for NGS library preparation comprises about 2.5 M NaCl and 20% Polyethylenglycol (PEG) 8000.

In inventive methods for the preparation of NGS libraries and the improved NGS workflow, the above-described buffer is added directly to the obtained RT-PCR amplification mixture containing the amplified nucleic acids. The inventive buffer is preferably added in ratio of 2:1 to 1:2 to the amplification mixture, and most preferably the inventive buffers are added in an about equal amount (e.g. 1:1) to the PCR amplification mixture. In a very preferred embodiment the inventive buffers for normalization beads for NGS library preparation comprises about 2.5 M NaCl and 20% Polyethylenglycol (PEG) 8000 are added in a ratio of 1:1 to the PCR amplification mixture.

The time and steps for the preparation of libraries for NGS can thus be strongly reduced. Further, the buffer added to the (RT-)PCR products is quite cheap, in particular it is much cheaper than the clean-up beads and the clean-up buffer.

The examples set out below serve only as examples and should by no means be construed as limiting the scope of the present invention.

### EXAMPLES

### Example 1

### Preparation of an HCV Library for NGS Using Vela Diagnostic's Automated Platform Sentosa SX101

### 1. RT-PCR

HCV viral RNA is isolated from human plasma and cDNA synthesized. Here, this step is performed using the automated platform Sentosa SX 101.
Before RT-PCR is conducted, Uracil-DNA-glycosylase (UDG) is added to the RT-PCR mix to eliminate potential contaminants derived from prior assays. Perform amplicon-carry over contaminant digestion with UDG for 4 min at 25° C. before amplification.
   2. Normalization after RT-PCR

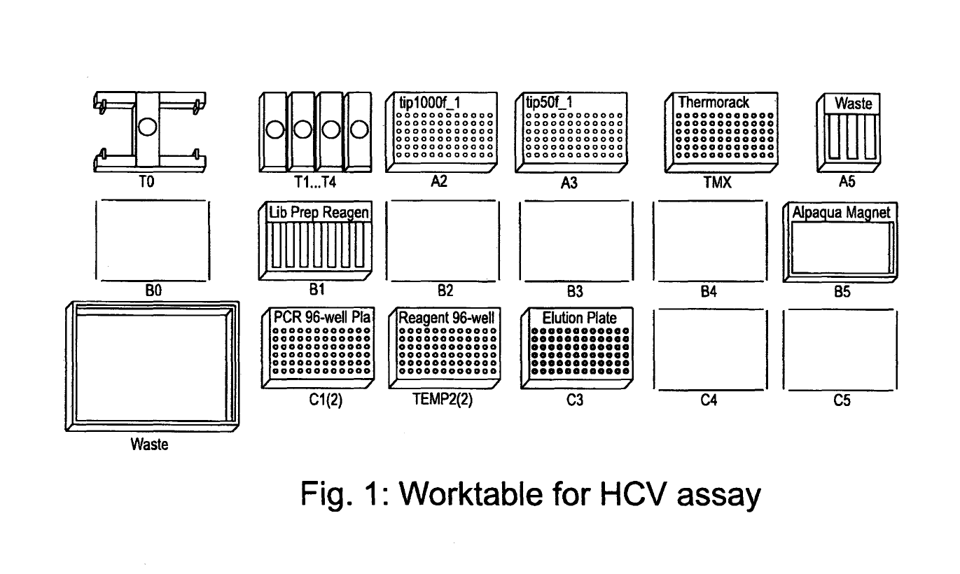

Reference is made to a working platform depicted in FIG. 1.

Prepare wells of Reagent 96-well plate (FIG. 1, position C1):

| Aliquot into the | Aliquot into Library |
|---|---|
| Reagent 96-well Plate | Prep Reagent holder |
| B4: 75 μL Shearase buffer | 1A: 220 μL Normalizer Beads |
| (Life technologies) | (Axygen) |
| C4: 50 μL Shearase enzyme | 1B: 500 μL Mineral Oil (Sigma) |
| D4: 50 μL Shearase enzyme | 1D: Empty Tube |
| B6: 30 μL dNTP mix | 2A: 1500 μL PCR clean-up buffer |
| C6: 100 μL 10X ligase buffer: | (Vela Diagnostics, Inc.) |
| D6: 50 μL DNA ligase | 2B: 15,000 μL Clean-up beads |
| E6: 30 μL Nick repair polymerase | (Axygen) |
| (Enzymatics) | 2C and 2D: Normalizer Binding |
| C8: P1 + barcode 12 mix | buffer (Axygen) |
| | 3A: 1500 μL Normalizer Elution |
| | buffer (Axygen) |
| | 3B and 3C: 1600 μL Nuclease free |
| | water (BST) |

Set temperature of the Reagent 96-well plate (TEMP2 in FIG. 1) to 15° C.;
Pool 25 μL of every PCR product (in the total of 4) of each sample to a defined position.
Mix 5× and transfer 195 μL of normalization beads to 1500 μL PCR clean-up buffer (Lib Prep Reagent);
Mix 10× and transfer 86 μL of PCR clean-up buffer (Vela Diagnostics) and Normalizer beads (Axygen). Mix (Lib Prep Reagent) to defined position of pooled PCR product and mix for 10 times.
Incubate for 3 min at room temperature;
Transport the PCR plate to the magnetic holder at position B5 in the platform in FIG. 1;
Incubate for 2 min;
Discard supernatant by pipetting 40 μL three times and 50 μL once;
Add 100 μL of 80% EtOH to selected well on the PCR plate;
Transfer PCR plate to the thermomixer (TMX) and shake at 1000 rpm for 2 min;
transport the PCR plate back to the magnetic holder at position B5 in FIG. 1;
The temperature control of the TMX is turned on and set to 56° C.;
Incubate for 2 min;
Discard supernatant by pipetting 70 μL three times and 40 μL once;
Transport the PCR plate to the thermomixer (TMX) set previously to 56° C.;
Dry the plate for 2 min;
Transport the PCR plate to position C1;
Add 35 μL of elution buffer (Lib Prep Reagent 1A to PCR Plate);
Mix 5 times by pipetting;
Transport the PCR plate to the thermomixer;
Shake for 5 min 1400 rpm at 56° C. on the thermomixer;
Transport plate back to the magnetic plate (B5) and wait for 2 min;

### 3. Shearing

Transfer 63 uL of shear buffer (Life technologies) and 30 uL to a defined position and mix 5 times. Transfer 80 ul of the mixture to shear enzyme (Life technologies) (C4 and D4) respectively mix 20×;
Transfer 15 ul to defined position. Transfer 15 uL of eluted sample (from step 2) to the same defined position and mix;
Transport PCR plate to the thermomixer and incubate 12 min, 38° C. for 13 minutes.

### 4. PCR Beads Clean-Up

Mix the PCR clean up beads and transfer 50 μL of the beads from Lib

Prep Reagent to defined PCR plate well;
Transport the PCR plate to TMX and shake at 1200 rpm for 3 min at 26° C.
Transport the PCR plate to the magnetic holder at position B5 and wait for 2 min.
Discard the supernatant by pipetting 70 μL and 30 uL respectively
Add 100 μL 80% EtOH (Lib Prep Reagent to PCR Plate);
Transport PCR plate back to the TMX and shake at 1200 rpm for 3 min at 26° C.;
Transport PCR plate to the magnetic holder at B5. Wait for 3 min;
Discard supernatant by pipetting 70 μL once and 50 μL once;
Dry beads by waiting for 5 min;
Transport the PCR plate back to location C1;
Add 28 μL elution buffer (transfer elution buffer from Lib Prep Reagent to selected PCR plate well);
Transport PCR plate to the TMX and shake at 1200 rpm for 2 min at 26° C.;
Transport PCR plate to magnetic holder on B5 and wait for 2 min;

### 5. Ligation

Transfer 90 μL of ligase buffer (Enzymatics), 18 uL dNTP, 36 uL T4 ligase (Enzymatics), 18 uL Manta polymerase (Enzymatics), and 108 uL of water from defined reagent plate well to another defined tube and mix by 10 times;
Transfer another 15 μL of the mix from Reagent plate defined well to another defined well;
Subsequently, transfer 10 uL of barcode adaptor to the same defined well.
Transfer 25 uL of sample eluted from step 4 to the same defined well and mix by ten times. Cover the mixture with 25 uL mineral oil.
Transport PCR plate to the TMX and incubate at 26° C. for 10 min;
Increase the temperature to 65° C. and incubate for another 5 min.

### Example 2

### AmpliSeg™ Library Automation

Prepare wells of Reagent 96-well plate (FIG. 1, position C1) using AmpliSeg™ reagents (Life technologies, Inc.):

| Aliquot the following into the | Aliquot the following into the Library |
|---|---|
| Reagent 96-well Plate | Prep Reagent holder (position B1) |
| A1: Primer pool 1: 10 μL | 1A: Elution buffer: 100 μL |
| C1: primer pool 2: 10 μL | 2A: Mineral oil: 300 μL |
| A3: PCR master mix: 15 μL | 2B: Binding buffer: 200 μL |
| A5: FuPa: 7 μL | 2C: Normalization beads: 40 μL |
| A7: Ligase enzyme: 7 μL | 2D: Nuclease-free water: 100 μL |
| C7: Switch solution: 15 μL | 6A: 80% ethanol: 2 mL |
| A9: Barcode adapter mix: 8 μL | |

Ampliseq library automation using automated platform Sentosa SX101 (Vela Diagnostics)

### 1. PCR

Set temperature at position TEMP2 to 4° C.
Transfer 4 μL of PCR master mix from defined wells in Reagent plate to primer pools in other selected well;
Mix by pipetting 10×;
Transfer 7 μL of PCR mix from selected Reagent 96-well Plate wells to selected PCR 96-well Plate wells, respectively;
Transfer 3 μL of gDNA samples from defined Elution Plate well to selected PCR 96-well Plate wells, respectively (Total PCR Vol.=10 μL);
Manually seal the PCR plate and transfer to the PCR for amplification using the following program:
Step 1: 99° C. 2 min
Step 2: 99° C. 15 sec
Step 3: 60° C. 4 min
repeat step 2 (21×)
Hold at 10° C.
After PCR, return PCR plate to C1 position on the Sentosa platform SX101 (FIG. 1);
Set thermomixer temperature to 52° C.

### 2. FuPa Reaction

Transfer 2 μL of FuPa (Life technologies) from selected Reagent 96-well Plate well to predetermined PCR 96-well Plate well. (Transferring of very small volumes of viscous reagents using an automated system);
Pool 10 μL of the PCR product from defined wells to well on the PCR plate that contains FuPa reagent and mix by pipetting 5×;
Add 40 μL oil overlay to PCR Plate well of previous step. (Lib Prep reagent->PCR Plate);
Transport the PCR Plate to the TMX and shake at 300 rpm at 52° C. for 10 min, 57° C. for 10 min, and 62° C. for 20 min;
Transfer the PCR Plate back to position C1 on the SX101.

### 3. Ligation Reaction

Add 4 μL of the Switch solution (Life technologies) from defined Reagent Plate well to other defined PCR Plate well;
Transfer 2 μL of ligase from defined Reagent Plate well to another defined PCR Plate well;
Transfer 2 μL of barcoded adapters from defined Reagent Plate well to predetermined PCR Plate well;
Add 5 μL of water from predetermined well containing Library Prep reagent to another predetermined PCR Plate well;
Add 17 μL sample and mix by pipetting 5×;
Transfer the entire sample from selected PCR Plate well to well which already contains the ligase and mix by pipetting 5×;
Add 40 μL oil overlay to selected PCR Plate well B5. (Lib Prep reagent to defined PCR Plate wells);
Wait for 20 min;
Set thermomixer to 72° C. and wait for another 10 min;
Transport the PCR Plate to the TMX and at 300 rpm at 72° C. for 10 min.

### 4. Bead Normalization

Mix normalization beads by pipetting for 10×;
Add 10 μL of normalization beads in Lib Prep Reagent to 200 μL of binding buffer in Lib Prep Reagent;
Transport the PCR Plate from the TMX to position C1;
Set the thermomixer temperature to 25° C.;
Mix the beads solution in defined well for 10× before transferring 100 μL of the beads solution to the desired PCR Plate well;
Transfer 25 μL of the sample from one selected PCR Plate well to another defined well for binding and mix by pipetting 10×;
Wait for 5 min;
Transport the PCR Plate to the TMX;
Shake at 1200 rpm for 1 min at 25° C.;
Incubate for 4 min;
Transport the PCR Plate to the magnetic plate holder B5 and incubate for 2 min;
Discard the supernatant by pipetting 50 μL for 2× and 20 μL for 1×;
Transfer 100 μL of 80% EtOH to selected PCR Plate well;
Transport the plate back to the TMX and shake at 1000 rpm for 1 min at 25° C.;
Incubate for 1 min;
Transport the PCR Plate back to the magnetic plate holder B5 and incubate for 2 min;
Discard the supernatant by pipetting 50 μL for 2× and 20 μL for 1×;
Transport the plate back to the TMX and shake at 1000 rpm for 1 min at 25° C.;
Incubate for 1 min;
Transport the PCR Plate back to the magnetic plate holder B5 and incubate for 2 min;
Discard the supernatant by pipetting 50 μL for 2× and 20 μL for 1×;
Set the TMX to 58° C.;
Transport the PCR plate to the TMX to dry off the EtOH for 2 min;
Transport the PCR plate back to position C1;
Add 25 μL of elution buffer to PCR Plate selected well;
Transport the PCR plate to the TMX and shake at 1200 rpm for 2 min;
Transport the PCR Plate to the magnetic plate holder B5 and incubate for 2 min;
Pipette 25 μL of the eluted sample from one defined PCR Plate well to another defined well.

The methods and additional aspects relating to such methods are less laborious, safe costs, reagents and are less prone to contamination than comparable methods that are not automated or require more manual steps.

1. A method of preparing a DNA library comprising the steps:
a) Extracting nucleic acids from a sample,
b) Exposing the extracted nucleic acids to a mixture comprising UDG, a DNA polymerase and optionally a reverse transcriptase, and dUTP,
c) Incubating the mixture to decontaminate the mixture from carry over amplification products derived from prior amplification reactions,
d) Performing an amplification reaction in the presence of dUTP,
wherein the decontamination reaction, optionally reverse transcription and DNA polymerase-conducted amplification reaction are performed in the same reaction mixture.

2. The method according to claim 1, wherein the DNA polymerase is a Thermus aquaticus (Taq) DNA polymerase, or a functional derivative thereof, wherein the functional derivative of Taq polymerase has at least 80%, preferably at least 90%, more preferably at least 100% of the DNA polymerization activity of Taq polymerase.

3. The method according to claim 1, wherein the extracted nucleic acids are fragmented prior to step b).

4. The method according to claim 1, wherein the DNA library is subsequently used in a next generation sequencing reaction.

5. A reagent composition comprising an enzyme mix comprising a UDG, a DNA polymerase, optionally a reverse transcriptase.

6. The reagent composition according to claim 5 further comprising dUTP.

7. The reagent composition according to claim 5 comprising Taq DNA Polymerase or a functional derivative thereof.

8. The reagent composition according to claim 5 comprising reagents for reverse transcription and/or PCR.

9. A method of decontaminating reaction mixture for the amplification of nucleic acid templates comprising said nucleic acid templates, a DNA polymerase, a UDG enzyme and optionally a reverse transcriptase, and dUTP, and reagents for DNA polymerization, and optionally reverse transcription.

10. The method according to claim 9, wherein the UDG enzyme is inactivated after a period sufficient to decontaminate the mixture from carry over amplification products derived from prior amplification reactions.

11. The method according to claim 9, wherein the DNA polymerase is a Thermus aquaticus (Taq) DNA polymerase, or a functional derivative thereof.

12. The method according to claim 9, wherein the extracted nucleic acids are fragmented prior to step b).

13. The method according to claim 9, wherein the DNA library is subsequently used in a next generation sequencing reaction.

14. A method for the preparation of a DNA library comprising the steps:
a) Extracting nucleic acids from a sample,
b) Exposing the extracted nucleic acids to a mixture comprising a DNA polymerase and optionally a reverse transcriptase,
c) Performing an amplification reaction in the presence of dUTP,
d) normalizing the obtained amplification products, wherein the normalization method comprises the following steps:
(i) adding a buffer composition comprising an alkali metal salt and a solvent to the amplification mixture comprising amplification products,
(ii) adding carrier particles to the amplification mixture comprising amplification products,
(iii) Incubating the mixture for a time sufficient for the DNA to bind to the carrier particles,
(iv) Washing the mixture with ethanol,
(v) Elution of normalized PCR products from the carrier particles.

15. The method according to claim 14, wherein the alkali metal salt is NaCl.

16. The method according to claim 14, wherein the solvent is polyethylene glycol.

17. The method according to claim 14, wherein the alkali metal salt is added in an amount of about 2.0 to about 5.0 M NaCl, preferably about 2.5 M NaCl.

18. The method according to claim 14, wherein the solvent is PEG 8000.
